(19) United States
(12) Patent Application Publication
(10) Pub. No.: US 2025/0192731 A1
(43) Pub. Date: Jun. 12, 2025

(54) AMPLIFIER DEVICE FOR AMPLIFYING SMALL CURRENTS

(71) Applicant: INFICON GMBH, Köln (DE)

(72) Inventor: Norbert Rolff, Horrem (DE)

(21) Appl. No.: 17/438,872

(22) PCT Filed: Mar. 6, 2020
(86) PCT No.: PCT/EP2020/056031

§ 371 Date: Mar. 2, 2022

(30) Foreign Application Priority Data
Mar. 29, 2019 (DE) 10 2019 108 192.0

(51) Int. Cl. H03F 1/52; G01N 33/00; G01R 19/00; H03F 3/45; H03F 3/72

(52) U.S. Cl. CPC H03F 1/523; G01N 33/0027; G01R 19/0092; H03F 3/45475; H03F 3/72; H03F 2200/261; H03F 2200/426; H03F 2200/444; H03F 2200/462; H03F 2203/45138

(57) ABSTRACT

An amplifier device for receiving currents, comprising a first current path for measuring smaller currents of less than 100 pA, an input amplifier device included in the first current path, having at least one first amplifier, an output, an inverting input, a first feedback path connecting the output to the inverting input, and a feedback element included in the first feedback path, wherein the first amplifier has at least one protective element, a second current path for measuring larger currents with a maximum current of at least 10 times the current to be received in the first current path, characterized in that at least one of the protective elements of the amplifier is part of the second current path.

The invention relates to a device and a method for amplifying small currents.

In many measuring devices, e.g. in mass spectrometers or vacuum measuring devices, the measured value is formed on the basis of currents which are generated by positively or negatively charged particles impinging on an electrode. The dynamic range of the occurring ion or electron current strength to be measured is very large. The dynamic range often ranges from a few hundred atto-amps to a few micro-amps, thus spanning several decades.

In particular, small currents are to be measured in vacuum measuring devices, in the lower pressure range and in leak detectors. Mass spectrometers are often used for this purpose. For very small currents below 10 fA, circuit difficulties arise when setting up an amplifier that can also measure larger currents at the same time, when range switching from measuring small currents to measuring larger currents is necessary. Current amplifiers using a resistor must include a high-impedance resistor for very small currents, since the current noise of the resistor decreases with increasing values. However, for currents greater than, for example, 10 pA, the high-impedance resistor cannot be used because the voltage drop across the resistor becomes too large. With capacitors as reference components, similar difficulties arise as with resistors. In amplifiers according to prior art, semiconductor switches or relays are used to switch resistors or current paths to enable switching from an operating state for measuring small currents to an operating state for measuring larger currents. In all cases, additional components are required for this purpose, which are connected to the sensitive input node at which the smallest currents flow and must be measured.

To measure such particle currents, measuring devices are used which generate a measurable measuring potential from the particle current, i.e. ion current or electron current. For this purpose, the measuring device usually has a current amplifier, typically with an operational amplifier. An electrical measuring resistor, arranged for example in the feedback path of the operational amplifier, is used to generate a measurable electrical potential from the amplified current.

In order to cover the entire dynamic range of the occurring particle currents with the same measuring circuit, both the conventional amplifiers and the amplifier according to the invention provide for switching between a sensitive operation for measuring small currents of less than 100 pA (e.g. 1 pA) and an operation for measuring larger currents of at least 1 pA (e.g. 100 pA). In conventional current amplifiers, the switching is done using a relay, for example, in sector field mass spectrometers. In the case of resistor-based current amplifiers covering a large current range, range switching cannot be dispensed with, since only high-impedance resistors can be considered for small currents, while for larger currents, only low-impedance components can carry the large current. For small currents, the resistance noise limits the range of application.

EP 0 615 669 B1 describes an amplifier in which the current signal is transmitted by means of diodes.

The object of the invention is to provide an amplifier device for amplifying small currents with which switching to amplify larger currents is improved, and to provide a corresponding method.

The amplifier device according to the invention is defined by independent claim 1.

According to the invention, the amplifier device has a first current path for amplifying small currents. The first current path includes an input amplifier device having at least one first amplifier with at least one protective element, e.g. a protective diode, and with a feedback element in a feedback path connecting the output of the input amplifier device to the inverting input. For measuring larger currents, a second current path at least partially different from the first current path is provided and formed. At least one of the protective elements of the first amplifier is included in the second current path.

Thus, according to the invention, an amplifier device is provided at whose input for measuring larger currents no further components are required than those for operation for measuring smallest currents, namely the current input, the measuring resistor in the form of the feedback element and the input amplifier device with at least the first amplifier. The conventionally used input amplifiers already have protective elements, e.g. protective diodes, in the input range, which are required in the integrated circuit to protect the input circuits. According to the invention, said protective elements are used for range switching. Thus, the most sensitive range for measuring the smallest currents of the circuit is as stable as if there were no other ranges for measuring other current quantities. When the range changes from the sensitive range to the range for measuring larger currents, the input current flows through one or more of the protective elements, making the input range low impedance even for much larger input currents. Since protective elements must be present in the integrated circuit of the first amplifier, not every amplifier module can be used. Both amplifier modules that have protective elements to the supply voltages and amplifier modules that have protective elements between the input lines can be used.

The input amplifier device can have a number of n amplifiers with n≥1. In the simplest case of n=1, the input amplifier device includes only the first amplifier. In the case of n≥2, the input amplifier device can be formed of at least two amplifiers forming an amplifier combination.

The input amplifier device has an inverting input and an output. The output is connected via a feedback path to the inverting input of the input amplifier device. A feedback element is included in the feedback path.

The amplifiers are operational amplifiers and/or the protective elements are protective diodes.

In the following, exemplary embodiments of the invention are explained in more detail with reference to the figures, in which

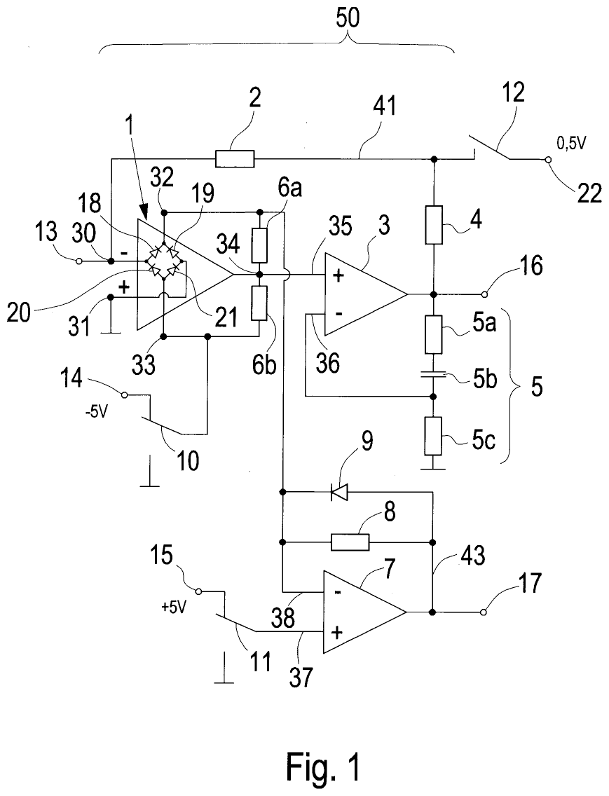

FIG. 1 shows the exemplary embodiment in a first operating state,

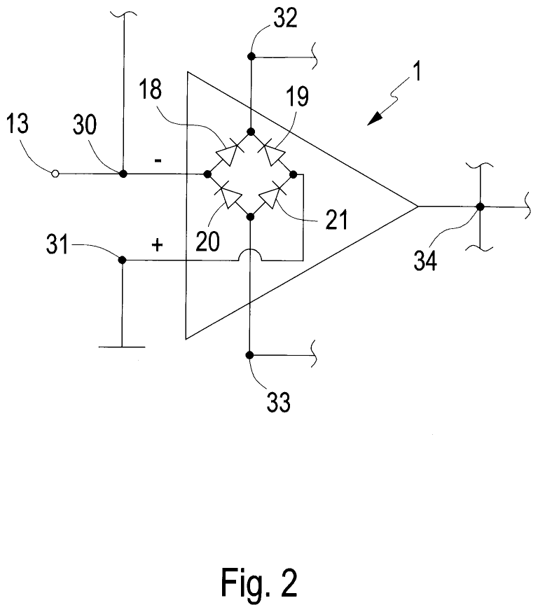

FIG. 2 shows a detail of FIG. 1,

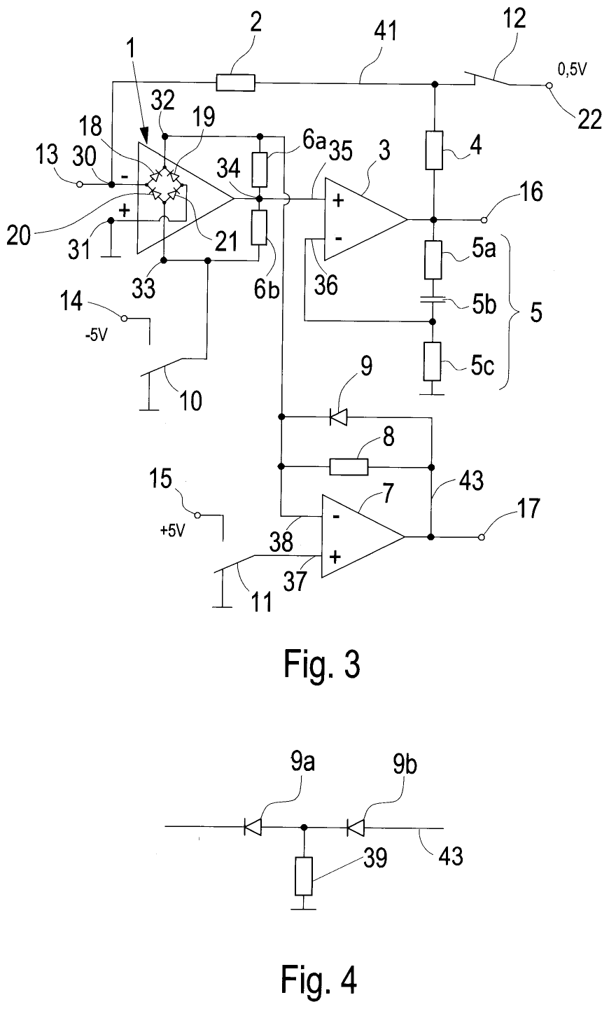

FIG. 3 shows the exemplary embodiment in a second operating state, and

FIG. 4 shows a detail of FIG. 1 or FIG. 3 according to a second exemplary embodiment.

The amplifier device of the illustrated exemplary embodiment comprises a first operational amplifier 1 having an inverting input 30, a non-inverting input 31, an output 34, a positive supply connection 32 and a negative supply connection 33.

The operational amplifier includes protective elements 18, 19, 20, 21, in this case diodes, which are arranged in the manner of a bridge rectifier circuit between inverting input 30 and non-inverting input 31 as well as the two supply connections 32, 33 and interconnect them. The diodes 18, 19, 20, 21 are arranged such that they conduct from negative supply connection 33 toward positive supply connection 32 and block in the opposite direction.

The expression “in the manner of a bridge rectifier circuit” means that the bridge branch includes the two inputs 30, 31 of operational amplifier 1. This is based on the idea that in an ideal operational amplifier no voltage is dropped between the two inputs 30, 31. One end of the bridge branch is connected to inverting input 30 and the other end to non-inverting input 31. The differential voltage occurring between the two inputs, which is zero in the ideal operational amplifier, is thus part of the bridge branch.

The output 34 of the first operational amplifier 1 is connected to non-inverting input 35 of a second operational amplifier 3 whose output is connected via a feedback branch 41 to inverting input 30 of the first operational amplifier 1. The feedback path 41 includes two resistors 2, 4 between which feedback path 41 is connected via a third switch 12 to an input 22 to which a voltage of a diode forward voltage of about 0.5 volts is applied.

The output 16 of the second operational amplifier 3 is further connected to ground via control elements 5. The control elements 5 consist of a resistor 5a, a capacitor 5b, and a further resistor 5c. The inverting input 36 of the second operational amplifier 3 is electrically connected for its feedback between capacitor 5b and the further resistor 5c.

The output 34 of the first operational amplifier 1 is connected via a first resistor 6a to positive supply connection 32 of the first operational amplifier 1 and via a second resistor 6b to negative supply connection 33. The negative supply connection 33 is alternately connectable via a first switch 10 to a supply voltage of −5 volts applied to voltage source 14 or ground 10.

The positive supply connection 32 of the first operational amplifier 1 is electrically connected to inverting input 38 of a third operational amplifier 7 whose output 17 is fed back via a resistor 8. The resistor 8 is bridged by a diode 9 which conducts in the direction from output 17 of the third operational amplifier 7 to positive supply connection 32 of the first operational amplifier 1 and blocks in the opposite direction.

The first amplifier 1 and the second amplifier 3 form an input amplifier device 50, wherein both amplifiers 1, 3 form an amplifier combination. The feedback path 41 connects the output of input amplifier combination 50 to its inverting input 30.

The non-inverting input 37 of the third operational amplifier 7 is selectively connectible via a second electric switch 11 to a voltage source 15, in this case 5 volts, or to ground.

In the illustrated exemplary embodiment of the amplifier device according to the invention, the first operation amplifier 1 is used as the input amplifier, the protective elements 18, 19, 20, 21 of which are provided as input protective diodes to the supply voltage. If the amplifier device is operated for measuring small currents, of, for example, less than 100 pA in the most sensitive range, the input current signal reaches the first operational amplifier 1 via input 13. The first operational amplifier 1 receives the negative supply voltage from voltage source 14 via the first switch 10 in the switch position shown in FIG. 1. The operational amplifier 1 receives the positive supply voltage from voltage source 15, in this case amounting to +5 volts, via the second switch 11 in the switch position shown in FIG. 1 at non-inverting input 37 of the third operational amplifier 7.

The output voltage at output 17 of the third operational amplifier 7 regulates itself to a value increased by the diode forward voltage of diode 9. As a result, the voltage reaching the first operational amplifier 1 becomes as large as the voltage at non-inverting input 37 of the third operational amplifier 7.

The amplifier 7 is not part of input amplifier device 50. The amplifier 7 serves for receiving larger current at input 13 of input amplifier device 50. The input current flows through diode 18 through amplifier 1 directly to input 38 of amplifier 7. Smaller currents at input 13, however, are first amplified by input amplifier device 50.

The third electric switch 12 is open in this operating state shown in FIG. 1 for measuring small currents. The feedback resistors 4, 2 together form the feedback element, in this case in the form of an electrical resistor.

In this case, input 13 at inverting input 30 of the first operational amplifier 1 represents a virtual zero point, which means that no significant voltage is present at input 13.

An inverted voltage proportional to the input current of the first operational amplifier 1 is applied to output 16 of the second operational amplifier 3. The protective diodes 18, 19, 20, 21 of the input amplifier are now each operated in the reverse direction and thus do not carry any appreciable current.

The input amplifier device 50 from the first operational amplifier 1 and the second operational amplifier 3 form a control circuit. The control elements 5 must be dimensioned depending of the cut-off frequency of the used elements 5a, 5b, 5c so that the circuit remains stable, e.g. 120 kOhm at electrical resistor 5a, 1 nF at capacitor 5b and 10 kOhm at electrical resistor 5c.

FIG. 1 shows the entire amplifier device, while FIG. 2 shows in detail the first operational amplifier 1 and the protective elements 18, 19, 20, 21 arranged therein and their electrical interconnection with the connections of the first operational amplifier 1.

FIG. 3 shows the amplifier device in the operating state for measuring larger currents via the second current path. For this purpose, the amplifier device is operated in an extended current range in which the first electric switch 10 is switched to ground potential. Thus, a voltage of approximately 0 volts is applied to negative supply connection 33 of the input amplifier. The input protective elements 20, 21 continue to block.

The second switch 11 is also switched to ground potential. Since the third operational amplifier 7 regulates itself with its feedback resistor 8 such that there is no appreciable differential voltage between inverting input 38 and non-inverting input 37 of the third operational amplifier 7, the positive supply of the first operational amplifier 1 at positive supply connection 32 will also be close to 0 volts. Now, when an input current is applied to input connection 13, it will flow through protective diode 18 to positive supply connection 32.

The third electric switch 12 is closed in this operating condition so that the voltage applied to connection 22, in this case 0.5 volts, is applied to feedback resistor 2 in accordance with the diode forward voltage of protective diode 18 so that as little current as possible flows through feedback resistor 2.

The second current path for larger currents at input 13 now leads via protective diode 18 to feedback resistor 8 of the third operational amplifier 7. An inverted voltage proportional to the input current is applied across feedback resistor 8, which can be tapped at output 17 of the third operational amplifier 7. In this case, diode 9 blocks. The second current path for measuring larger currents then leads from inverting input 30 of the first operational amplifier 1 via a protective diode 18 of the protective diodes 18, 19, 20, 21 to positive supply connection 32 of the first operational amplifier 1 and from there via feedback resistor 8 of the third operational amplifier 7 to its output connection 17.

In the described circuit arrangement, input voltage 13 is increased by the forward voltage of protective element 18 when operating in the current path for larger currents. To avoid this characteristic, the ground points on switches 10 and 11 can be lowered by the forward voltage of protective element 18. In this case, feedback element 2 is currentless when 220 volts are applied to the input.

A further improvement of the second current path results when diode 9 is divided according to FIG. 4. In the case where the second current path has been activated, a negative voltage is applied to output 17. The reverse current of the diode falsifies the measurement. If the circuit is complemented according to FIG. 4, the negative voltage at diode 9b drops. Approximately zero volts are then applied to both sides of diode 9a, which significantly reduces the reverse current.

The circuit for positive input currents has been described here. In principle, a circuit variant for negative or also both current directions is possible.

1. An amplifier device for receiving currents, comprising
a first current path for measuring small currents of less than 100 pA,
an input amplifier device (50) included in the first current path, comprising
at least one first amplifier (1),
an output,
an inverting input (30),
a first feedback path (41) connecting the output to the inverting input (30), and
a feedback element (2) included in the first feedback path (41),
wherein the first amplifier (1) has at least one protective element (18, 19, 20, 21),
a second current path for measuring larger currents with a maximum current of at least 10 times the current to be received in the first current path,
characterized in that,
at least one of the protective elements (18, 19, 20, 21) of the input amplifier device (50) is part of the second current path.

2. The amplifier device according to claim 1, characterized in that the supply voltage of the first amplifier (1) is applied to at least one of the protective elements (18, 19, 20, 21).

3. The amplifier device according to any one of the preceding claims, characterized in that at least one of the protective elements (18, 19, 20, 21) is arranged between two inputs (30, 31) of the first amplifier (1) and connected thereto.

4. The amplifier device according to any one of the preceding claims, characterized in that the feedback element (2) in the first feedback path (41) is an electrical resistor.

5. The amplifier device according to any one of the preceding claims, characterized in that the feedback component (2) in the first feedback path (41) is a capacitor.

6. The amplifier device according to any one of the preceding claims, characterized in that the input amplifier device (50) comprises at least one further amplifier (3) forming a control loop with the first amplifier (1).

7. The amplifier device according to any one of the preceding claims, characterized in that at least one of the two supply connections (32, 33) of the first amplifier (1) is connected via an electrical resistor (6a, 6b) to the output of the first amplifier (1).

8. The amplifier device according to any one of the preceding claims, characterized in that the feedback path (41) connects the output of the last amplifier of the input amplifier device (50) consisting of the first amplifier (1) and the at least one further amplifier (3) to the inverting input (30) of the input amplifier device (50).

9. The amplifier device according to any one of the preceding claims, characterized in that the negative supply connection (33) of the first amplifier (1) is selectively connectible via a first switch (10) to a negative supply voltage (14) or ground, while the positive supply connection (32) of the first amplifier (1) is electrically conductively connected to the inverting connection (38) of a third amplifier (7), the non-inverting input (37) of which is selectively connectible via a second switch (11) to a positive supply voltage (15) or ground.

10. The amplifier device according to any one of the preceding claims, characterized in that the third amplifier (7) comprises a second feedback path (43) with an electrical resistor (8) and an electrical diode (9) arranged in parallel with the resistor (8) and conducting in the direction from the output (17) of said third amplifier to the positive supply connection (32) of the first amplifier (1) and blocking in the opposite direction.

11. The amplifier device according to any one of the preceding claims, characterized in that the first feedback path (41) is selectively connectible via a third switch (12) to a voltage which avoids or allows current flow in the feedback element (2) depending on the switch position.

12. The amplifier device according to any one of the preceding claims, characterized in that the ground references of the switches (10, 11) and the reference voltage (22) are changed by the forward voltage of the protective element (18).

13. The amplifier device according to any one of the preceding claims, characterized in that the second feedback path (43) comprises two diodes (9a, 9b) between which a resistor (39) leads with respect to the switch (11).

14. A gas detector comprising a mass spectrometric sensor or a total pressure sensor and an amplifier device according to any one of the preceding claims.

15. A method for measuring currents by using an amplifier device according to any one of claims 1-13, characterized in that switching from the first current path to the second current path takes place by connecting the negative supply connection (33) of the first amplifier (1) to ground.

16. The method according to the preceding claim, characterized in that, for switching the current path, the non-inverting input of a third amplifier (7), whose inverting input is connected to the positive supply connection (32) of the first amplifier (1), is additionally connected to ground.
